(19) United States
(12) Patent Application Publication
(10) Pub. No.: US 2026/0114949 A1
(43) Pub. Date: Apr. 30, 2026

(54) Container for Medical Devices and Associated Systems and Methods

(71) Applicant: Avent, Inc., Alpharetta, GA (US)

(72) Inventor: Brett C. Leard, Seneca, SC (US)

(21) Appl. No.: 19/003,285

(22) Filed: Dec. 27, 2024

Related U.S. Application Data
(60) Provisional application No. 63/616,002, filed on Dec. 29, 2023.

(51) Int. Cl. A61B 50/30; A61B 50/00

(52) U.S. Cl. CPC A61B 50/30; A61B 2050/0053

(57) ABSTRACT

A container for a medical device is provided, the container comprising a lid portion, a base portion, and a joining panel. The lid portion comprises lid inner and outer panels opposing and spaced apart each other, a lid front panel extending between a front edge of the lid inner panel and a front edge of the lid outer panel, and a lid back panel extending from a back edge of the lid inner panel toward a back edge of the lid outer panel. The base portion is pivotably coupled to the lid portion and comprises similarly arranged inner and outer panels, a front panel, and back panels. The joining panel extends between the back edge of the lid outer panel and the back edge of the base outer panel. Also provided herein are methods for preparing said container and dispensing said medical device.

### CROSS-REFERENCE TO RELATED APPLICATIONS

This application claims the benefit of U.S. Provisional Application No. 63/616,002, filed December 29, 2023, the contents of which is incorporated herein by reference in its entirety.

### BACKGROUND

Medical devices and medical device accessories increasingly contain sensitive components, such as carefully calibrated electronic sensors or delicate mechanical structures. It is possible for vibrations incurred during transit and handling of such devices to interfere with their functionality. Additionally, medical devices must often be removed from sterile packaging before they are used in a patient. The process of removal can itself lead to poorly controlled release and/or otherwise introduce unfavorable vibrations or unsterile conditions to the device. As such, a need exists for packaging that can accommodate medical devices and medical device accessories that helps to reduce vibrations, maintain sterility of the contents, and increase the ease and stability of dispensing of the device.

### SUMMARY

Objects and advantages of the present disclosure will be set forth in part in the following description, or may be obvious from the description, or may be learned through practice of the invention.

In some aspects, the present disclosure is directed to a container for a medical device comprising: a lid portion comprising: a lid inner panel; a lid outer panel, spaced apart from and opposing the lid inner panel; a lid front panel extending between a front edge of the lid inner panel and a front edge of the lid outer panel; a lid back panel extending from a back edge of the lid inner panel toward a back edge of the lid outer panel; a base portion pivotably coupled to the lid portion, the base portion comprising: a base inner panel; a base outer panel, spaced apart from and opposing the base inner panel; a base front panel extending between a front edge of the base inner panel and a front edge of the base outer panel; a base back panel extending from a back edge of the base inner panel toward a back edge of the base outer panel; and a joining panel extending between the back edge of the lid outer panel and the back edge of the base outer panel; wherein the lid portion and/or the base portion are moveable between a closed configuration and an open configuration, where in the closed configuration the lid inner panel is positioned adjacent to the base inner panel, and in the open configuration the lid inner panel is spaced apart from the base inner panel.

In some examples, in the closed configuration, the lid back panel is coplanar with the base back panel.

In some examples, in the closed configuration, each of the lid back panel and base back panel are substantially parallel with the joining panel.

In some examples, in the closed configuration, each of the lid back panel and base back panel are in contact with the joining panel.

In some examples, in the closed configuration, the back edge of the lid inner panel is colinear with the back edge of the base inner panel.

In some examples, in the closed configuration, the back edge of the lid inner panel and the back edge of the base inner panel are in contact with the joining panel.

In some examples, in the closed configuration, the lid front panel is coplanar with the base front panel.

In some examples, in the closed configuration, the lid outer panel is substantially parallel with the base outer panel.

In some examples, in the open configuration, the lid front panel is not coplanar with the base front panel.

In some examples, in the open configuration, the lid outer panel is not parallel with the base outer panel.

In some examples, when the container moves from the closed configuration toward the open configuration: an angle between the lid outer panel and the joining panel increases, and an angle between the base outer panel and the joining panel increases.

In some examples, when the container moves between the closed configuration and the open configuration: the lid back panel pivots with respect to the joining panel at the lid outer panel back edge; and the base back panel pivots with respect to the joining panel at the base outer panel back edge.

In some examples, when the container moves from the closed configuration toward the open configuration: an angle between the lid back panel and the joining panel increases, and an angle between the base back panel and the joining panel increases.

In some examples, in the open configuration, the lid back panel and base back panel are not coplanar.

In some examples, in the open configuration, neither the lid back panel nor the base back panel are substantially parallel with the joining panel.

In some examples, in the open configuration, neither the back edge of the lid inner panel and the back edge of the base inner panel are in contact with the joining panel.

In some examples, in the open configuration, the back edge of the lid inner panel and the back edge of the base inner panel are spaced apart.

In some examples, when in the open configuration, the lid inner panel and the lid outer panel are spaced apart; and the base inner panel and the base outer panel are spaced apart.

In some examples, in both the open and closed configuration, the lid back panel is coplanar with the base back panel.

In some examples, in both the open and closed configuration, each of the lid back panel and base back panel are substantially parallel with the joining panel.

In some examples, in both the open and closed configuration, each of the lid back panel and base back panel are in contact with the joining panel.

In some examples, in both the open and closed configuration, the back edge of the lid inner panel is colinear with the back edge of the base inner panel.

In some examples, in both the open and closed configuration, each of the back edge of the lid inner panel and the back edge of the base inner panel are in contact with the joining panel.

In some examples, when the container moves from the closed configuration toward the open configuration, a spacing between the lid outer panel and the lid inner panel decreases.

In some examples, when the container moves from the closed configuration toward the open configuration, a spacing between the base outer panel and the base inner panel decreases.

In some examples, when the container moves between the closed configuration and the open configuration, the lid inner panel and the base inner panel pivot with respect to each other along the back edge of the lid inner panel and the back edge of the base inner panel, wherein the back edge of the lid inner panel and the back edge of the base inner panel are colinear.

In some examples, when the container moves between the closed configuration and the open configuration, the lid front panel pivots with respect to the lid inner panel and the lid outer panel.

In some examples, when the container moves between the closed configuration and the open configuration, the base front panel pivots with respect to the base inner panel and the base outer panel.

In some examples, when in the open configuration such that the lid outer panel and base outer panel are coplanar: the lid inner panel is positioned adjacent the lid outer panel; and the base inner panel is positioned adjacent the base outer panel.

In some examples, when the container moves from the closed configuration toward the open configuration: an angle between the lid front panel and the lid outer panel at the front edge of the lid outer panel decreases, and an angle between the lid front panel and the lid inner panel at the front edge of the lid inner panel increases.

In some examples, when the container moves from the closed configuration toward the open configuration: an angle between the lid back panel and the lid outer panel at the back edge of the lid outer panel increases, and an angle between the lid back panel and the lid inner panel at the back edge of the lid inner panel decreases.

In some examples, when the container moves from the closed configuration toward the open configuration: an angle between the base front panel and the base outer panel at the front edge of the base outer panel decreases, and an angle between the base front panel and the base inner panel at the front edge of the base inner panel increases.

In some examples, when the container moves from the closed configuration toward the open configuration: an angle between the base back panel and the base outer panel at the back edge of the base outer panel increases, and an angle between the base back panel and the base inner panel at the back edge of the base inner panel decreases.

In some examples, the base inner panel defines a base opening sized and configured to receive a medical device, the base inner panel comprising a first tab extending from a first side edge of the base opening and a second tab extending from a second side edge of the base opening, wherein the first tab and the second tab extend from the base inner panel in a direction toward the base outer panel.

In some examples, the first tab and the second tab of the base inner panel provide an inwardly directed (e.g., directed toward a center of the base opening) force and an upwardly directed (e.g., directed toward the base inner panel) force holding the medical device within the base opening and urging the medical device away from the base outer panel.

In some examples, the base inner panel comprises a first locking tab extending from a first locking side edge of the base opening, wherein the first locking tab extends from the base inner panel in a direction toward the lid inner panel.

In some examples, the base inner panel comprises a second locking tab extending from a second locking side edge of the base opening, wherein the second locking tab extends from the base inner panel in a direction toward the lid inner panel.

In some examples, the first locking side edge is on an opposite side of the base opening relative to the second locking side edge.

In some examples, a medical device, wherein the first locking tab and the second locking tab provide an inwardly directed (e.g., directed toward a center of the base opening) force and a downwardly directed (e.g., directed toward the base outer panel) force holding the medical device within the base opening.

In some examples, the medical device comprises at least one of a medical device accessory (e.g., an accessory coupling between a syringe and needle, such as a pressure differential sensor).

In some examples, the base inner panel defines a first finger opening adjacent the first side edge or second side edges of the base.

In some examples, a portion of the first finger opening extends beyond the first side edge or second side edge and into the first tab or second tabs of the base.

In some examples, the lid inner panel defines a lid opening sized and configured to receive a medical device, the lid inner panel comprising a first tab extending from a first side edge of the lid opening and a second tab extending from a second side edge of the lid opening, wherein the first tab and the second tab extend from the lid inner panel in a direction toward the lid outer panel.

In some examples, the first tab and the second tab of the lid inner panel provide an inwardly directed (e.g., directed toward a center of the lid opening) force and a downwardly directed (e.g., directed toward the lid inner panel) force holding the medical device within the lid opening and urging the medical device away from the lid outer panel.

In some examples, the lid inner panel defines a second finger opening adjacent the first side edge or second side edges of the lid.

In some examples, a portion of the second finger opening extends beyond the first side edge or second side edge and into the first tab or second tabs of the lid.

The present disclosure is further directed to a method of dispensing a medical device from a container.

In some examples, the method comprises providing a container with a medical device retained therein, the container comprising: a lid portion comprising: a lid inner panel; a lid outer panel, spaced apart from and opposing the lid inner panel; a lid front panel extending between a front edge of the lid inner panel and a front edge of the lid outer panel; and a lid back panel extending from a back edge of the lid inner panel toward a back edge of the lid outer panel; a base portion pivotably coupled to the lid portion, the base portion comprising: a base inner panel defining a base opening sized and configured to receive the medical device; a base outer panel, spaced apart from and opposing the base inner panel; a base front panel extending between a front edge of the base inner panel and a front edge of the base outer panel; and a base back panel extending from a back edge of the base inner panel toward a back edge of the base outer panel; and a joining panel extending between the back edge of the lid outer panel and the back edge base outer panel.

In some examples, moving the container from a closed configuration toward and open configuration comprises moving the lid portion and/or the base portion from a closed configuration toward and open configuration, where in the closed configuration the lid inner panel is positioned adjacent to the base inner panel, and in the open configuration the lid inner panel is spaced apart from the base inner panel.

In some examples, the method comprises decreasing a spacing between the base outer panel and the base inner panel such that a spacing between the top surface of the medical device and the base inner panel increases.

In some examples, the base inner panel further comprises a first tab extending from a first side edge of the base opening and a second tab extending from a second side edge of the base opening, wherein, when the container moves from the closed configuration toward the open configuration, the first and second tabs urge the medical device away from the base outer panel, wherein, decreasing a spacing between the base outer panel and the base inner panel causes the base outer panel to exert an upward force on a portion of the medical device and/or the first tab or the second tab such that the medical device is released from within the base opening.

In some examples, the base inner panel further comprises a first locking tab extending from a first locking side edge of the base opening and a second locking tab extending from a second locking side edge of the base opening, wherein, when the base outer panel exerts an upward force on a portion of the medical device and/or the first tab or the second tab, the medical device exerts an upward force against the first locking tab and the second locking tab, and the medical device is released from within the base opening.

In some examples, the container moves from a closed configuration toward and open configuration: the lid back panel pivots with respect to the joining panel at the lid outer panel back edge; and the base back panel pivots with respect to the joining panel at the base outer panel back edge.

In some examples, in the open configuration, a spacing between the base outer panel and the base inner panel decreases and the medical device is released from the base opening.

In some examples, the medical device comprises a central opening extending therethrough, wherein, in the open configuration, the central opening is provided above the base inner panel.

The present disclosure is further directed to a method for packaging and sterilizing a medical device and container.

In some examples, the method comprises the steps of: (1) providing a container comprising: a lid portion comprising: a lid inner panel; a lid outer panel, spaced apart from and opposing the lid inner panel; a lid front panel extending between a front edge of the lid inner panel and a front edge of the lid outer panel; a lid back panel extending from a back edge of the lid inner panel toward a back edge of the lid outer panel; and a base portion pivotably coupled to the lid portion, the base portion comprising: a base inner panel; a base outer panel, spaced apart from and opposing the base inner panel; a base front panel extending between a front edge of the base inner panel and a front edge of the base outer panel; and a base back panel extending from a back edge of the base inner panel toward a back edge of the base outer panel; and a joining panel extending between the back edge of the lid outer panel and the back edge base outer panel, wherein the lid portion and/or the base portion are moveable between a closed configuration and an open configuration, where in the closed configuration the lid inner panel is positioned adjacent to the base inner panel, and in the open configuration the lid inner panel is spaced apart from the base inner panel; (2) with the lid portion and/or the base portion in an open configuration, inserting a medical device into a base opening provided in the base inner panel; (3) retaining the medical device with the base opening by engaging a first tab extending from a first side edge of the base opening with the medical device, the first tab providing an inwardly directed (e.g., directed toward the center of the base opening) force and an upwardly directed (e.g., directed toward the base inner panel) force against the medical device; (4) moving the container into a closed configuration; (5) inserting into the container into a pouch; (6) sealing the pouch; and (7) sterilizing the pouch.

In some examples, retaining the medical device with the base opening further comprises engaging a first locking tab extending from a first locking side edge of the base opening with the medical device, the first locking tab providing an inwardly directed (e.g., directed toward a center of the base opening) force and a downwardly directed (e.g., directed toward the base outer panel) force holding the medical device within the base opening.

In some examples, retaining the medical device with the base opening further comprises engaging a second locking tab extending from a second locking side edge of the base opening with the medical device, the second locking tab providing an inwardly directed (e.g., directed toward a center of the base opening) force and a downwardly directed (e.g., directed toward the base outer panel) force holding the medical device within the base opening.

In some examples, the method further comprises inserting the pouch into an outer container. In some examples, the outer container can include a plurality of pouches, for example, 10 pouches per container.

In some examples, moving the container into the closed configuration comprises securing the container in the closed configuring using an engagement mechanism between the lid portion and the base portion (e.g., tab and opening, tape, adhesive, etc.).

These and other features, aspects, and advantages of the present disclosure will become better understood with reference to the following description and appended claims. The accompanying drawings, which are incorporated in and constitute part of this specification, illustrate examples of the claimed invention and, together with the description, serve to explain the principles of the disclosure.

### BRIEF DESCRIPTION OF THE DRAWINGS

FIG. 1 provides a perspective view of a container for a medical device according to an example of the present subject matter.

FIG. 2 provides a left side view of the container of FIG. 1 containing a medical device in the closed configuration according to an example of the present subject matter.

FIG. 3 provides a right side view of the container of FIG. 1 containing a medical device in the closed configuration according to an example of the present subject matter.

FIG. 4 provides a right side perspective view of the container of FIG. 1 according to an example of the present subject matter.

FIG. 5 provides a right side perspective view of the container of FIG. 1 in the open configuration according to an example of the present subject matter.

FIG. 6 provides a top view of the container of FIG. 1 in the open configuration according to an example of the present subject matter.

FIG. 7 provides a top view of the container of FIG. 1 in the open configuration containing a medical device according to an example of the present subject matter.

FIG. 8 provides a right side perspective view of the container of FIG. 1 containing a medical device according to an example of the present subject matter.

FIG. 9 provides a right side perspective view of the container of FIG. 1 and a dispensed medical device according to an example of the present subject matter.

FIG. 10 provides a right side perspective view of the container of FIG. 1 and a dispensed medical device according to an example of the present subject matter.

FIG. 11 provides a right side perspective view of the container of FIG. 1 and a dispensed medical device according to an example of the present subject matter.

FIG. 12 provides a top plan view of the blank of the container of FIG. 1 according to an example of the present subject matter.

FIG. 13 provides a top view of a container for a medical device view of the container in the open configuration according to an example of the present subject matter.

FIG. 14 provides a right side view of the container of FIG. 13 in the closed configuration according to an example of the present subject matter.

FIG. 15 provides a right side view of the container of FIG. 13 in the closed configuration containing a medical device according to an example of the present subject matter.

FIG. 16 provides a right side perspective view of the container of FIG. 13 containing a medical device according to an example of the present subject matter.

FIG. 17 provides a right side perspective view of the container of FIG. 13 and a dispensed medical device according to an example of the present subject matter.

FIG. 18 provides a right side perspective view of the container of FIG. 13 and a dispensed medical device according to an example of the present subject matter.

FIG. 19 provides a top plan view of the blank of the container of FIG. 13 according to an example of the present subject matter.

Like reference symbols in the various drawings indicate like elements.

### DETAILED DESCRIPTION

Reference will now be made in detail to one or more examples of the invention, examples of which are illustrated in the drawings. Each example is provided by way of explanation of the invention and is not meant as a limitation of the invention. For example, features illustrated or described as part of one example may be used with another example to yield still a further example. It is intended that the invention include these and other modifications and variations as coming within the scope and spirit of the invention.

Before explaining at least one example of the invention in detail, it is to be understood that the invention is not limited in its application to the details of construction and the arrangement of the components set forth in the following description or illustrated in the drawings. The invention is capable of other example or of being practiced or carried out in various ways. Also, it is to be understood that the phraseology and terminology employed herein is for the purpose of description and should not be regarded as limiting.

### System

The present disclosure relates generally to packaging for containing and dispensing a medical device or medical device accessory, such as, for example, the “pressure sensing device” for intra-articular needle placement provided in US 11,690,868. The container may be one aspect of a packaging system/assembly for medical devices and/or medical device accessories.

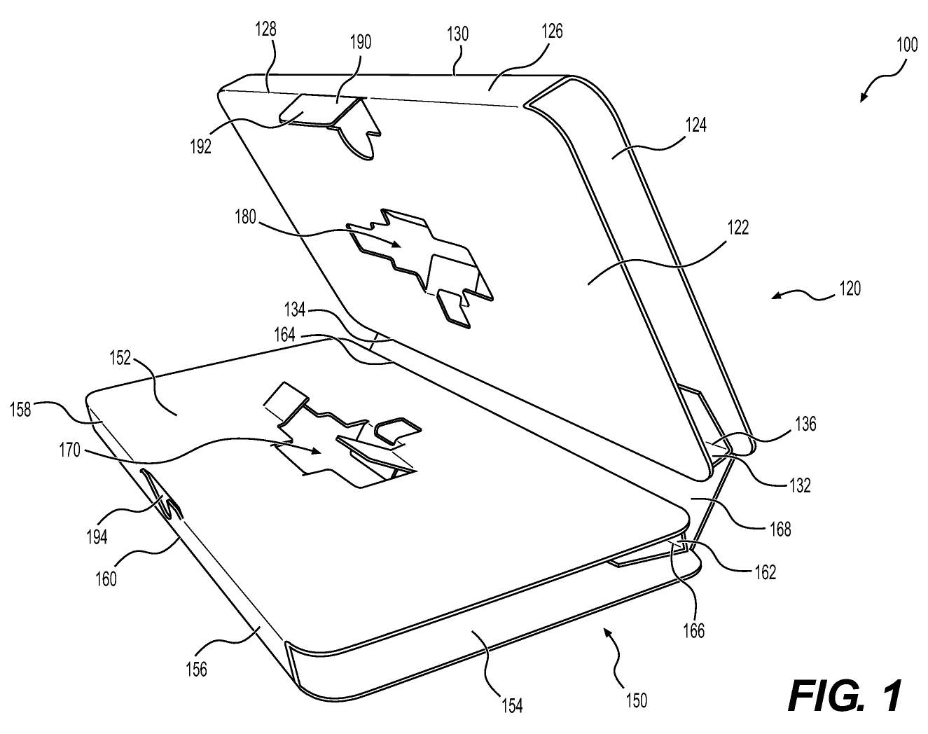

Referring now to the drawings, FIG. 1 illustrates a perspective view of an example container 100 for a medical device 200 of the present disclosure. As shown, the container 100 includes a lid portion 120, a base portion 150, and a joining panel 168 connecting the lid portion 120 with the base portion 150. The lid portion 120 includes a lid inner panel 122, a lid outer panel 124 spaced apart from and opposing the lid inner panel 122. A lid front panel 126 extends between a front edge 128 of the lid inner panel 122 and a front edge 130 of the lid outer panel 124, and a lid back panel 132 extends from a back edge 134 of the lid inner panel 122 toward a back edge 136 of the lid outer panel 124. The base portion 150 is pivotably coupled to the lid portion 120. In some examples, the base portion 150 is pivotably coupled to the lid portion 120 via the joining panel 168. The base portion 150 includes a base inner panel 152, a base outer panel 154 spaced apart from and opposing the base inner panel 152. A base front panel 156 extends between a front edge 158 of the base inner panel 152 and a front edge 160 of the base outer panel 154, and a base back panel 162 extends from a back edge 164 of the base inner panel 152 toward a back edge 166 of the base outer panel 154. As illustrated in FIG. 1, the joining panel 168 extends between the back edge 136 of the lid outer panel 124 and the back edge 166 of the base outer panel 154.

### Open/Closed Configuration

The container 100 is movable between a closed configuration and an open configuration. In the closed configuration, the lid inner panel 122 of the lid portion 120 the is positioned adjacent to the base inner panel 152 of the base portion 150, and in the open configuration the lid inner panel 122 is spaced apart from the base inner panel 152. In some examples, the spacing between the base outer panel 154 and the base inner panel 152 may be decreased such that a spacing between the top surface 202 of the medical device 200 and the base inner panel 152 increases. For example, the base outer panel 154 and/or the base inner panel 152 may bend, flex, or otherwise deform such that the space between the base inner panel 152 and the base outer panel 154 decreases. The medical device 200 can be constructed from a more rigid material such that as the space between the base inner panel 152 and base outer panel 154 decreases, the medical device 200 is, at least partially, released from the container 100, and the base inner panel 152 moved away from the top surface 202 of the medical device 200. In some examples described herein, the container 100 may be further configured to dispense a medical device 200 automatically upon movement from the closed configuration toward the open configuration and/or the container 100 may be configured to dispense the medical device 200 thereafter upon additional movement of the container 100.

As described herein, the lid portion 120 and/or the base portion 150 of the container 100 are moveable between a closed configuration and an open configuration. For example, FIG. 1 shows the lid portion 120 and/or the base portion 150 of the container 100 in an at least partially open configuration.

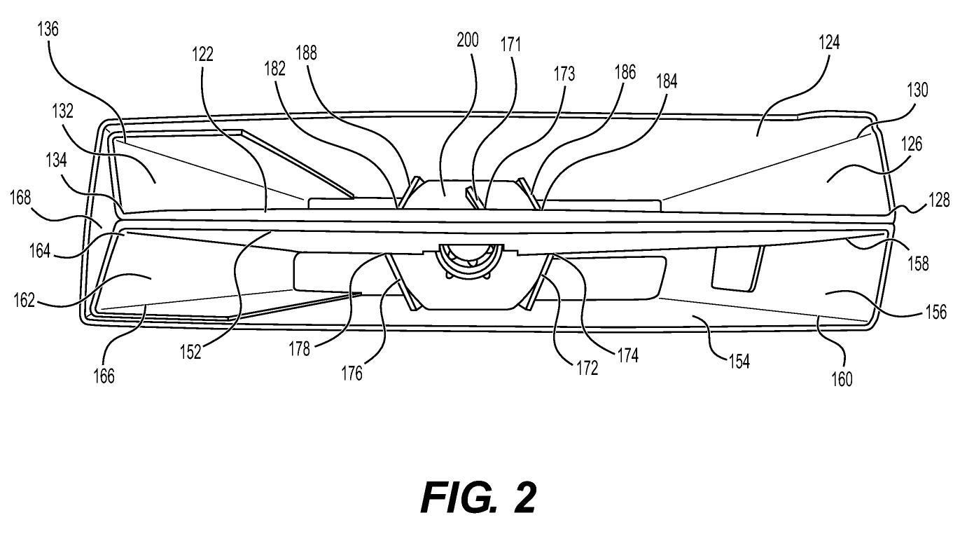

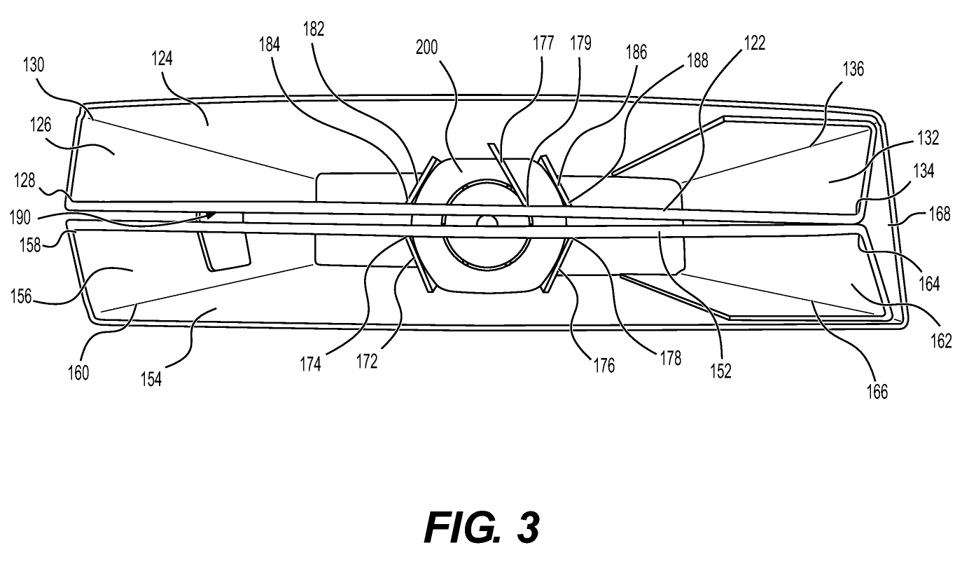

Referring now to FIGS. 2-3, the container 100 is shown in the closed configuration. In the closed configuration, the lid inner panel 122 is positioned adjacent to the base inner panel 152. In some examples, a portion of the lid inner panel 122 may be in direct contact with a portion of the base inner panel 152. In some examples, the lid outer panel 124 is substantially parallel with the base outer panel 154. As illustrated in FIG. 2, in the closed configuration, the lid back panel 132 and the base back panel 162 are proximate the joining panel 168. In some examples, each of the lid back panel 132 and base back panel 162 may be substantially parallel with the joining panel 168. In some examples, the lid back panel 132 and the base back panel 162 contact the joining panel 168 when the container 100 is in the closed configuration. As shown in FIG. 2, the back edge 134 of the lid inner panel 122 are colinear with the back edge 164 of the base inner panel 152. In some examples, the back edge 134 of the lid inner panel 122 and the back edge 164 of the base inner panel 152 may contact the joining panel 168. As shown in FIG. 2, in the closed configuration, the front edge 128 of the lid front panel 126 is colinear with the front edge 158 of the base front panel 156. In some examples, the lid front panel 126 is aligned and/or coplanar with the base front panel 156.

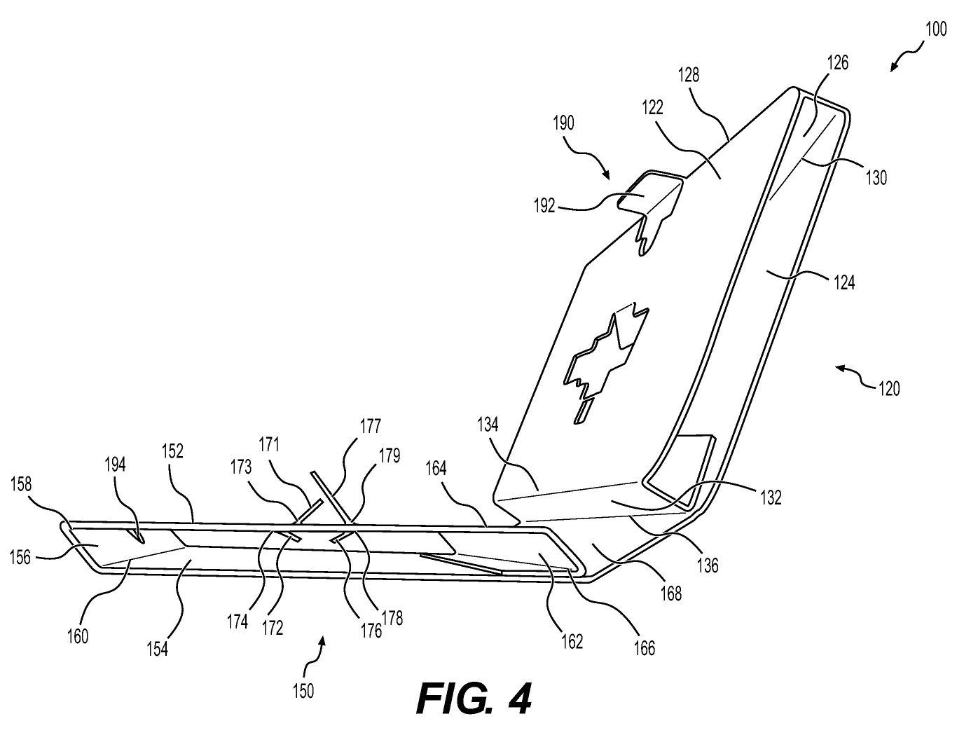

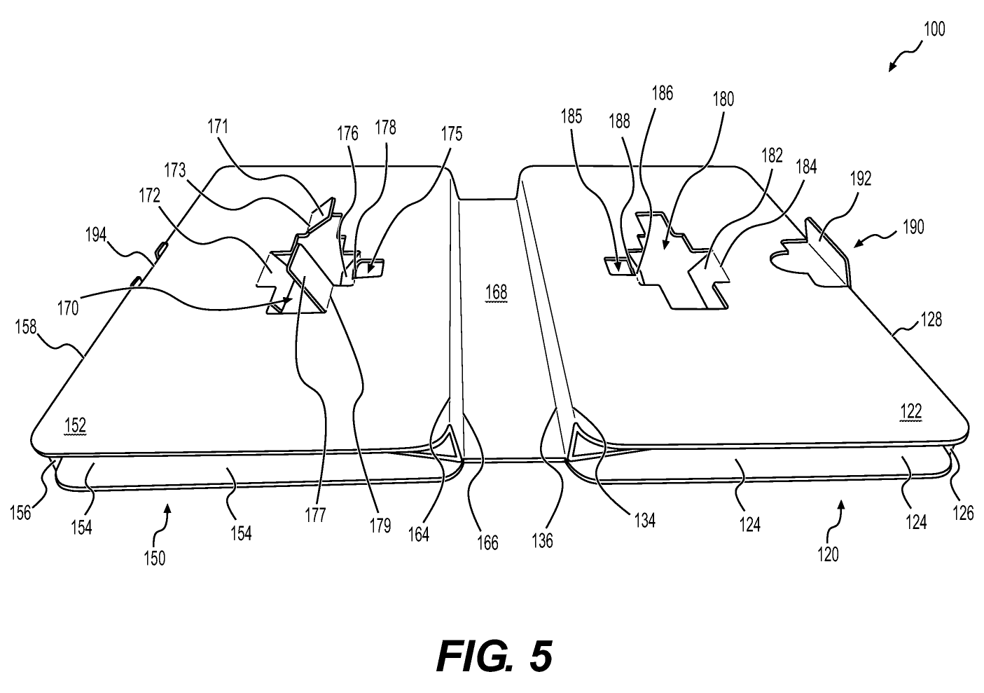

FIGS. 4-5 show the container 100 in the open configuration. FIG. 4 shows the container 100 in a partially open configuration, and FIG. 5 shows the container 100 in a fully open configuration. In some examples, as shown by comparing the closed container 100 of FIGS. 2- 3 to open container 100 of FIGS. 4-5, when the container 100 moves from the closed configuration toward the open configuration, the angle between the lid outer panel 124 and the joining panel 168 increases, and an angle between the base outer panel 154 and the joining panel 168 increases such that the lid inner panel 122 is spaced apart from the base inner panel 152. As shown in FIGS. 4-5, spacing the lid inner panel 122 from the base inner panel 152 generally results from pivoting movement of the lid inner panel 122 relative to the base inner panel 152 about the back edge 136 of the lid outer panel 124 and the back edge 166 of the base outer panel 154. As further shown in FIGS. 4-5, as the relative spacing between the lid inner panel 122 and the base inner panel 152 increases, the lid front panel 126 moves relative to the base front panel 156 such that they are no longer aligned and/or coplanar.

### Base Opening and Tabs

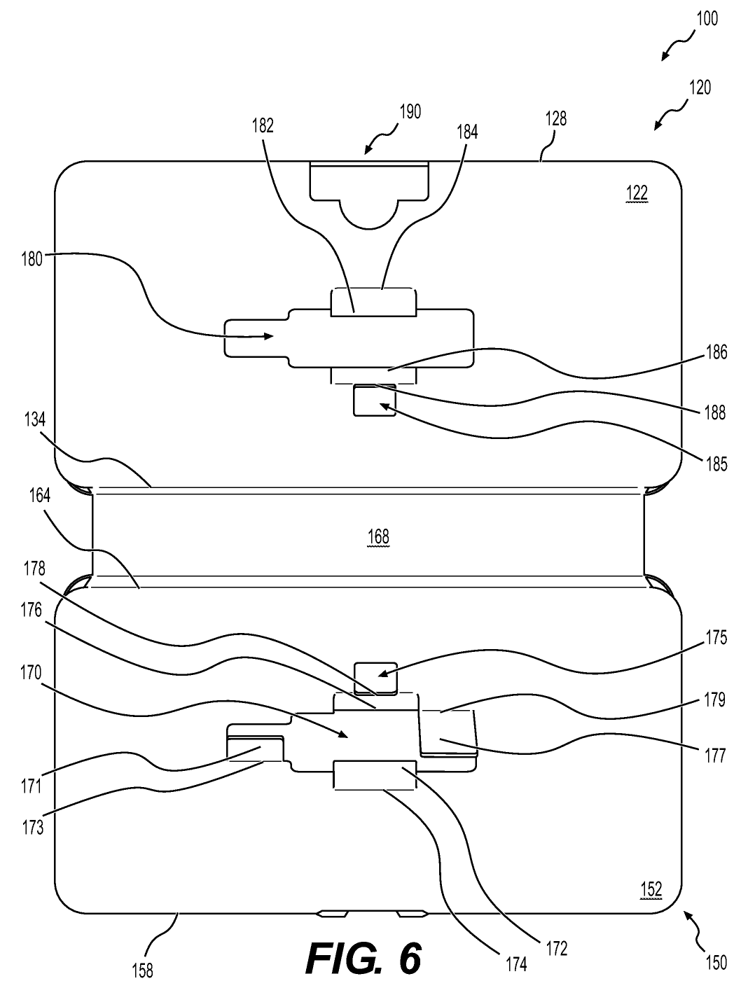

As described herein, the container 100 is sized and configured to accommodate and secure the medical device 200 therewithin. Accordingly, the container 100 may include various openings and tabs for accommodating and securing the medical device 200 within the container 100. In some examples, as shown in FIGS. 5-6, the base inner panel 152 defines a base opening 170 sized and configured to receive the medical device 200. For instance, as shown in FIGS. 2-3 and 7, the medical device 200 may include a medical device accessory. For example, medical device 200 may include a medical device accessory that couples between a syringe and needle. In a particular embodiment, shown in FIGS. 2-3 and 7, the medical device as a pressure differential sensor for improved intra-articular needle placement.

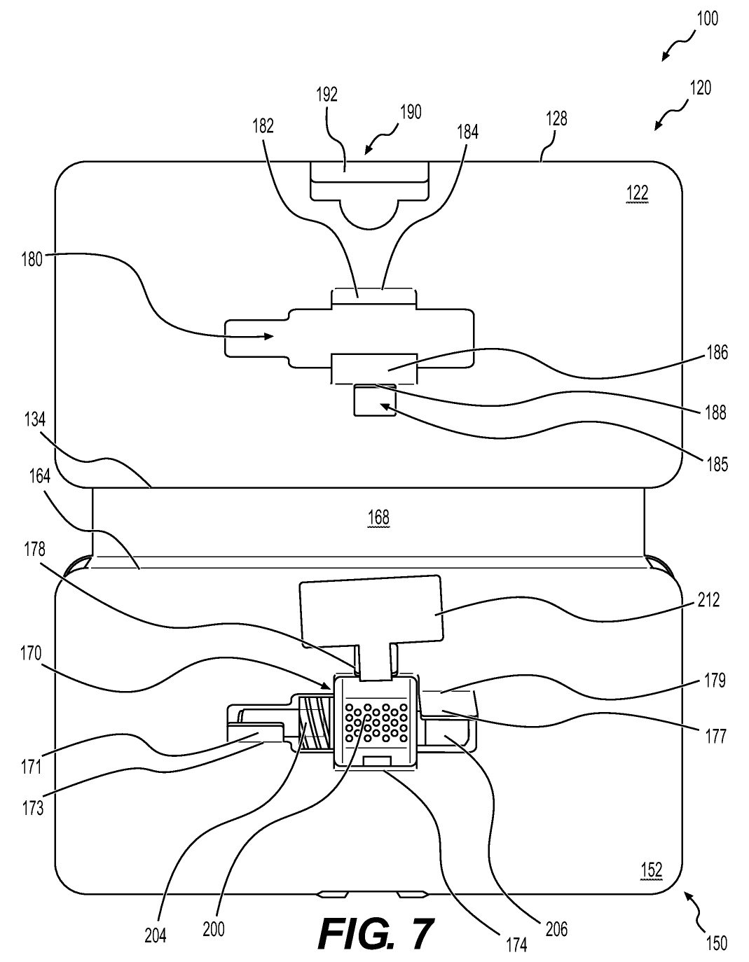

In some examples, the base opening 170 may have a size, shape, and location on the base inner panel 152 (and/or lid inner panel 122) corresponding with the size, shape, and desired location of the medical device 200 with respect to the container 100. For example, FIG. 7 shows the base opening 170 shaped to receive a medical device 200 having first and second luer locks 204, 206.

In some examples, as shown in FIGS. 5-6, the base inner panel 152 includes a first tab 172 extending from a first side edge 174 of the base opening 170 and a second tab 176 extending from a second side edge 178 of the base opening 170. In some examples, as shown in FIGS. 2-3, both the first tab 172 and the second tab 176 extend from the base inner panel 152 in a (inward) direction toward the base outer panel 154. In this configuration, the first tab 172 and second tab 176 provide an inwardly directed (e.g., directed toward a center of the base opening 170) and upwardly directed (e.g., directed toward the base inner panel 152) force against the medical device 200 received within the base opening 170. By supplying these forces, the first tab 172 and second tab 176 hold the medical device 200 within the base opening 170 and urge the medical device 200 away from the base outer panel 154. In some examples, the first tab 172 and second tab 176 prevent and/or reduce the likelihood that the medical device 200 will contact the base outer panel 154, as such contact could cause vibrations that could interfere with the function of and/or damage the medical device 200.

In some examples, as best shown in FIGS. 2 and 4, the base inner panel 152 also includes a first locking tab 171. The first locking tab 171 extends from the first locking side edge 173 of the base opening 170 in a (upward/outward) direction toward the lid inner panel 122. Furthermore, as best shown in FIGS. 3-4, the base inner panel 152 also includes a second locking tab 177. The second locking tab 177 extends from the second locking side edge 179 of the base opening 170 in a (upward/outward) direction toward the lid inner panel 122. As shown, the first locking side edge 173 may be on an opposite side of the base opening 170 relative to the second locking side edge 179 such that the first locking tab 171 is opposite the second locking tab 177 relative to the base opening 170/medical device 200. In examples that include one or both of the first locking tab 171 and second locking tab 177, the tabs provide an inwardly directed force (e.g., directed toward a center of the base opening) and a downwardly directed force (e.g., directed toward the base outer panel 154) so as to hold the medical device 200 within the base opening 170. For example, FIG. 7 shows a particular example where the first locking tab 171 and second locking tab 177 apply inwardly and downardly directed forces on the first and second luer locks of a medical device 200, securing the medical device 200 within the base opening 170.

### Openings and Tabs

Referring again to FIGS. 5-6, the lid inner panel 122 also defines a lid opening 180 sized and configured to receive the medical device 200. In some examples, the lid opening 180 may be sized, shaped, and located on the lid inner panel 122 to correspond with location and features of the medical device 200. For example, FIG. 7 shows the lid inner panel 122 sized and shaped to receive a medical device 200 having first and second luer locks 206, 208.

In some examples, as shown in FIGS. 5-6, the lid inner panel 122 may include a first tab 182 extending from a first side edge 184 of the lid opening 180 and a second tab 186 extending from a second side edge 188 of the lid opening 180. In some examples, as shown in FIGS. 2-3, both the first tab 182 and the second tab 186 extend from the lid inner panel 122 in a (inward) direction toward the lid outer panel 124 to provide inwardly directed (e.g., directed toward a center of the lid opening 180) and downwardly directed (e.g., directed toward the lid inner panel 122) forces on the medical device 200. By supplying these forces, the first tab 182 and second tab 186 hold the medical device 200 within the lid opening 180 and urge the medical device 200 away from the lid outer panel 124. Thus, the first tab 182 and second tab 186 reduce the likelihood that the medical device 200 will contact the base outer panel 124. Such contact could cause vibrations that could interfere with the function of and/or damage the medical device 200.

In some examples, as best shown in FIGS. 2-3, the lid opening 180 may further define openings sized and configured to receive the first locking tab 171 and second locking tab 177 when the container 100 is in the closed configuration.

In some examples, the container 100 includes an engagement mechanism 190 for releasably engaging the lid portion 120 with the base portion 150. The engagement mechanism 190 may include, for example, one or more tabs and corresponding openings, tape, adhesive, or any other means of releasably coupling the lid portion 120 and base portion 150. As shown in FIG. 1, for example, the engagement mechanism 190 may include a tab 192 extending from front edge 128/lid front panel 126 of the lid inner panel 122 that engages a corresponding opening 194 in front edge 158/base front panel 156 of the base inner panel 152.

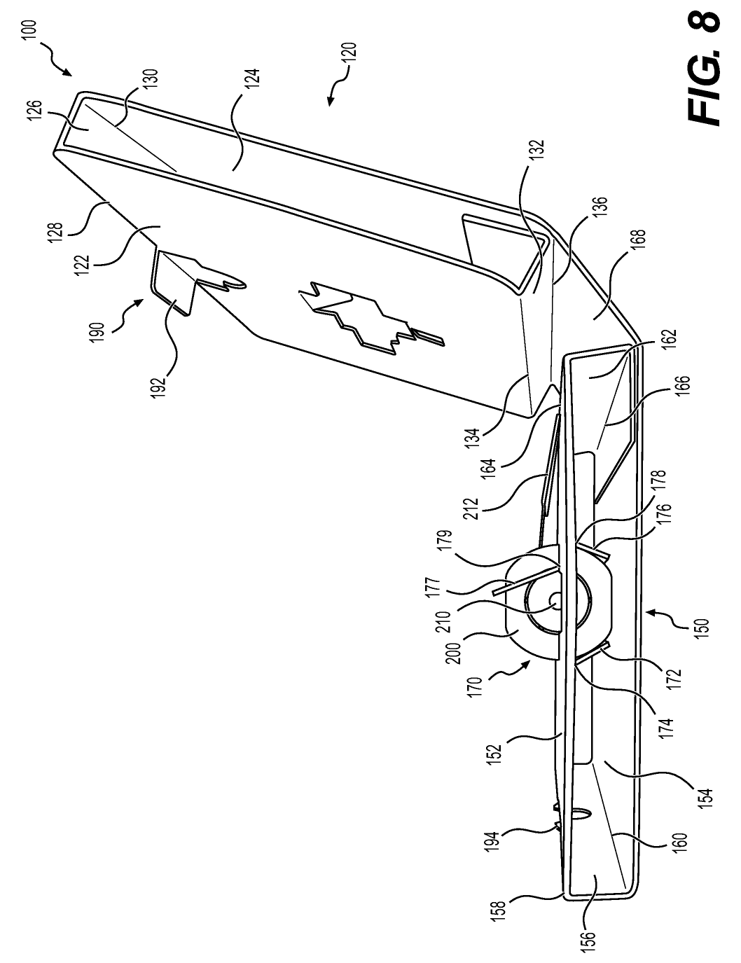

In some examples, as shown in FIGS. 7-8, the medical device 200 may include a pull tab 212. Specifically, as shown in FIG. 8, the pull tab 212 may not be vertically centered along the medical device 200. As a result, when the medical device 200 is received within the base opening 170, the pull tab 212 is spaced apart from the base inner panel 152. It is possible that the pull tab 212 could urge against the closing of the lid portion 120 as the container 100 is moved from the open to the closed configuration, thereby resisting secure engagement of the engagement mechanism 190. Therefore, in some examples, the opening 194 allows the tab 192 to flex therethrough when in the container 100 is in the closed configuration, thereby permitting secure engagement of the engagement mechanism 190.

By supplying the above-described forces, the first tab 172 and second tab 176 of the base inner panel 152 and the first tab 182 and second tab 186 of the lid inner panel 122 hold the medical device 200 within the base opening 170 and lid opening 180. Advantageously, this configuration enables the various tabs to accommodate vibrations that may occur during transport or handling of the container 100, thereby cradling the medical device 200 and reducing potentially harmful contact or vibrations that could interfere with its function and prevent possible damage to the medical device 200.

In some examples, the base inner panel 152 and lid inner panel 122 may also define first and second finger openings 175 and 185, respectively. The first finger opening 175 may be disposed adjacent the first side edge 174 or second side edge 178 of the base inner panel 152. In some examples, the first finger opening 175 extends beyond either side edge 174, 178 and into the first tab 172 or second tab 176. In a particular example, shown in FIGS. 5-6, the first finger opening 175 is disposed adjacent the second side edge 178 of the base inner panel 152 and extends into the second tab 176.

The second finger opening 185 may be disposed adjacent the first side edge 184 or second side edge 188 of the lid inner panel 122 and may extend beyond either side edge 174, 188 and into the first tab 182 or second tab 186. In a particular example, shown in FIGS. 5-6, the second finger opening 185 is disposed adjacent the second side edge 188 of the lid inner panel 122 and extends into the second tab 182.

### Example 1: Aspects Specific to Primary Embodiment

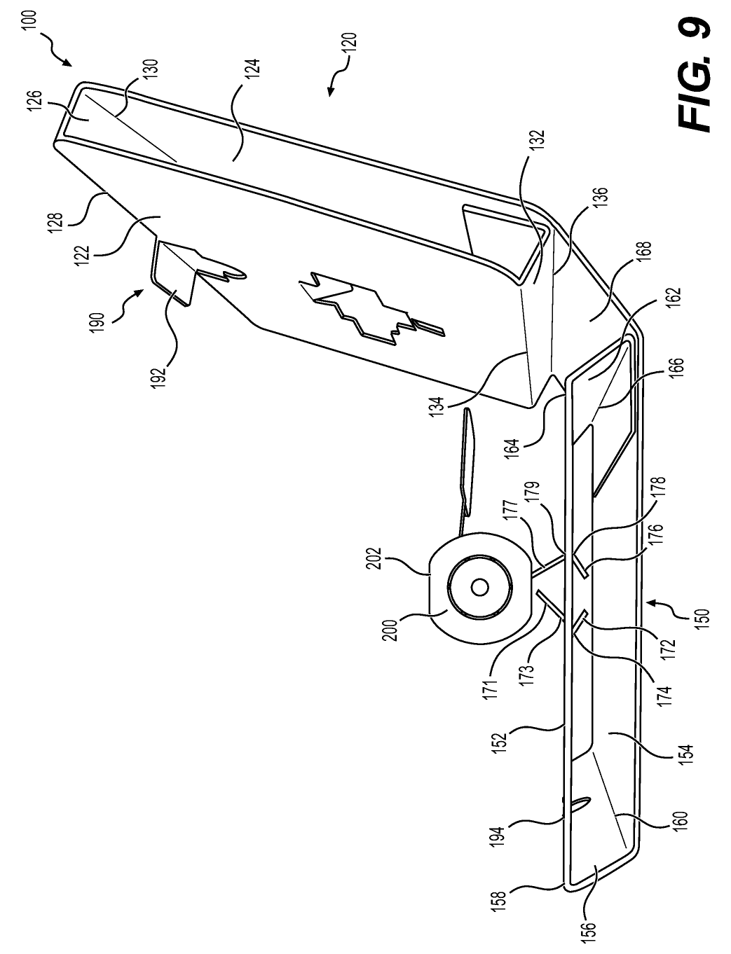

In the example container 100 shown in FIGS. 1-12, the container 100 can move from the closed configuration toward the open configuration without automatically dispensing the medical device 200. As best shown in FIGS. 3-5, when the container 100 moves between the closed configuration and the open configuration, the lid back panel 132 pivots with respect to the joining panel 168 at the back edge 136 of the lid outer panel 124, and the base back panel 162 pivots with respect to the joining panel 168 at the back edge 166 of the base outer panel 154. As shown, each of the lid back panel 132 and base back panel 162 are able to pivot with respect to the joining panel 168 independent of each other.

In such examples, as shown in FIGS. 3-5, when moving the container 100 toward the open configuration, the angle between the lid back panel 132 and the joining panel 168 increases, and the angle between the base back panel 162 and the joining panel 168 also increases. As shown, the joining panel 168 is interposed between the lid outer panel 124 and the base outer panel 154 so as to form a gap between the lid and base outer panels 124, 154. Because the lid back panel 132 and the base back panel 162 are not fixed relative to the joining panel 168, moving the container 100 toward the open configuration causes the lid and base back panels 132, 162 to not be coplanar. Rather, when moving from the closed configuration toward the open configuration, the back edge 134 of the lid inner panel 122 and the back edge 164 of the base inner panel 152 may separate from each other and be spaced apart from the joining panel 168. Thus, in some examples, in the open configuration, neither the lid back panel 132 nor the base back panel 162 are substantially parallel with or in contact with the joining panel 168. As shown in FIG. 5, in the open configuration, the lid inner panel 122 and the lid outer panel 124 are spaced apart, and the base inner panel 152 and the base outer panel 154 are spaced apart.

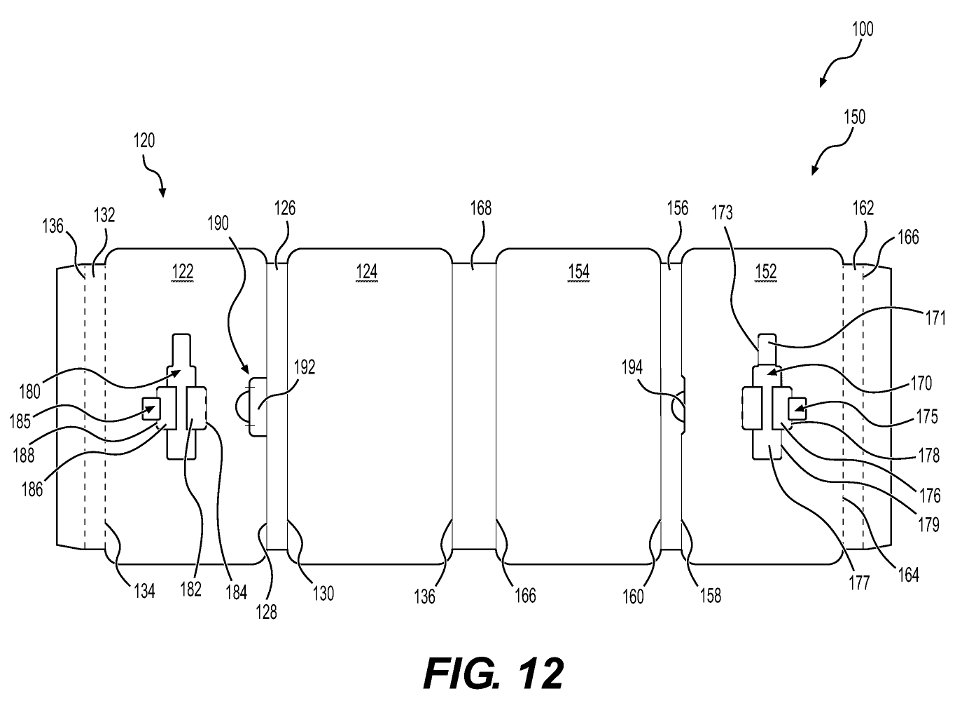

As shown in FIGS. 7-8, the above-described spacing between the base inner panel 152 and the base outer panel 154 permit the medical device 200 to rest within the base opening 170 even when the container 100 is moved toward the open configuration. FIG. 12 provides a dieline/blank of an exemplary embodiment of the container 100, according to the implementation shown in FIGS. 1-11.

### Example 2: Aspects Specific to Secondary Embodiment

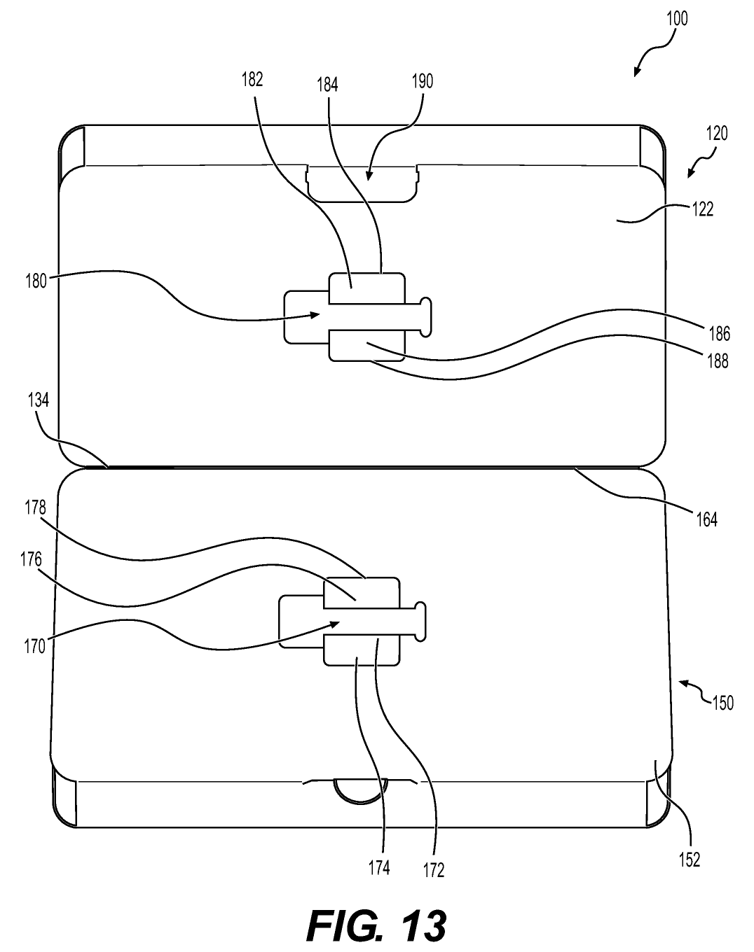

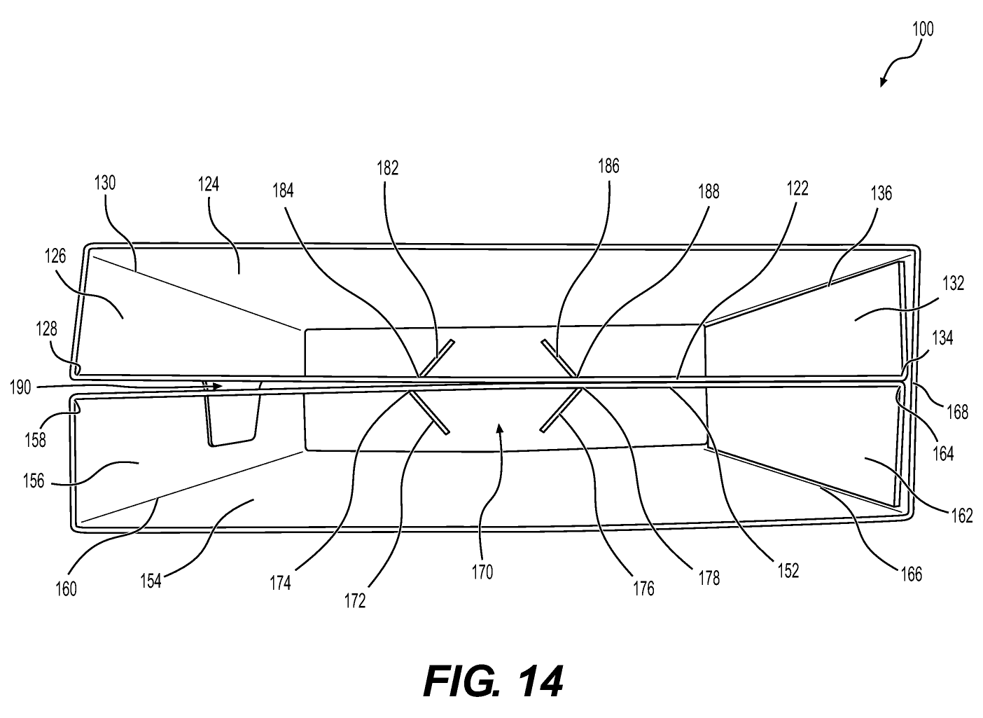

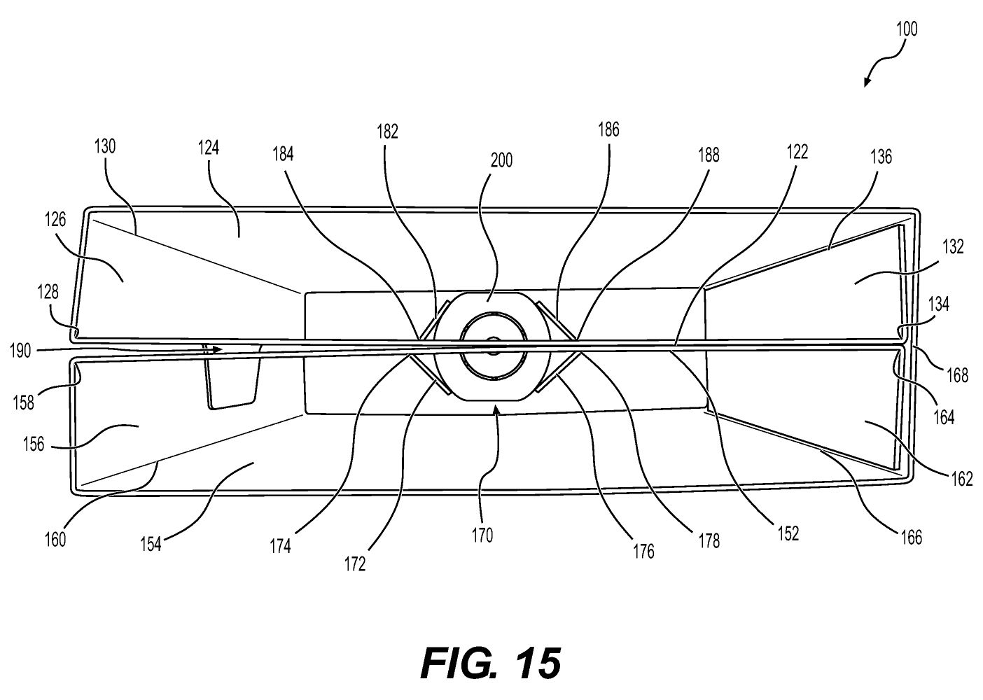

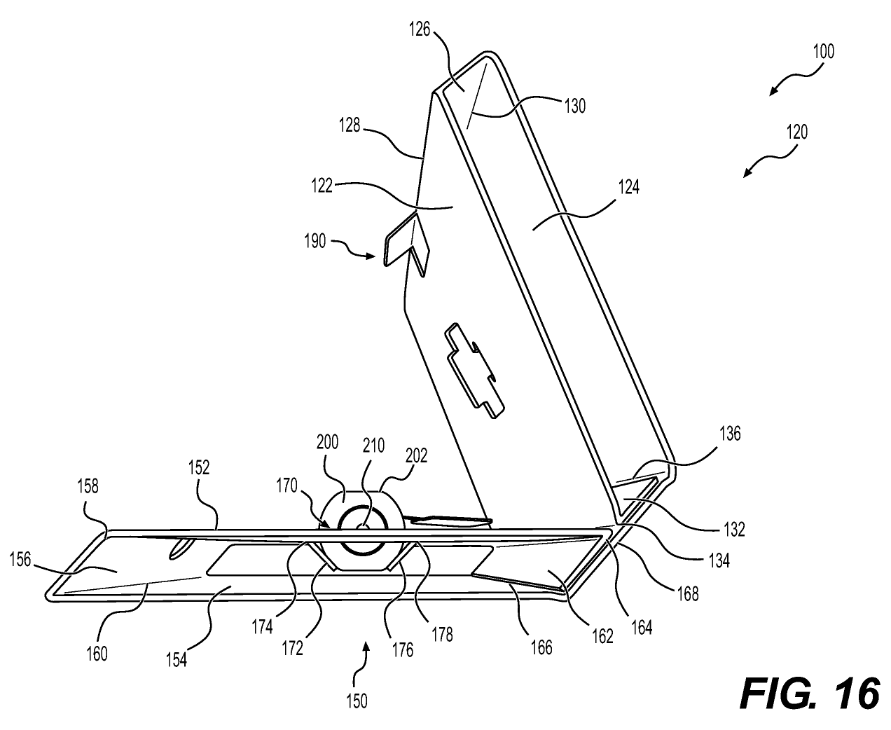

In another example container as shown in FIGS. 13-19, the container 100 is configured to automatically dispense the medical device 200 as the container 100 moves from the closed configuration toward the open configuration. As best shown in FIGS. 15-18, in both open and closed configurations, the lid back panel 132 remains coplanar with the base back panel 162, both the lid and base back panels 132, 162 are substantially parallel with the joining panel 168. In some examples, both the lid and base back panels 132, 162 are in contact with the joining panel 168 in both the open and closed configurations. Additionally, as shown, the back edge 134 of the lid inner panel 122 and the back edge 164 of the base inner panel 152 are colinear and also in contact with the joining panel 168 in both the open and closed configurations.

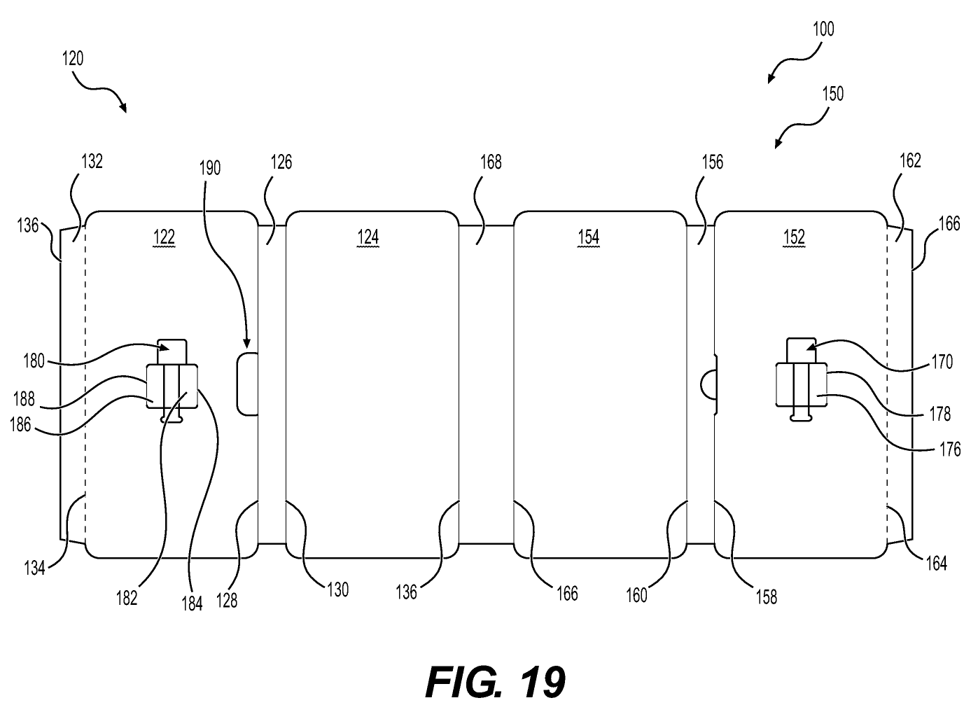

As shown in FIGS. 15-18, when the container 100 moves between the closed configuration and the open configuration, the lid inner panel 122 and the base inner panel 152 pivot with respect to each other along the back edge 134 of the lid inner panel 122 and the back edge 164 of the base inner panel 152, although the back edge 134 of the lid inner panel 122 and the back edge 164 of the base inner panel 152 remain colinear. As such, the lid front panel 126 pivots with respect to the lid inner panel 122 and the lid outer panel 124 and the base front panel 156 pivots with respect to the base inner panel 152 and the base outer panel 154. FIG. 19 provides a dieline/blank of an exemplary embodiment of the container 100, according to the implementation shown in FIGS. 13-18.

When in the open configuration such that the lid outer panel 124 and base outer panel 154 are coplanar, the lid inner panel 122 is positioned adjacent the lid outer panel 124 and the base inner panel 152 is positioned adjacent the base outer panel 154. In fact, the lid inner panel 122 may contact the lid outer panel 124 and the base inner panel 152 may contact the base outer panel 154.

As described above, as the container 100 moves from the closed configuration toward the open configuration, various panels pivot relative to each other. Accordingly, various angles formed between the panels also change.

With respect to the lid portion 120, as shown in the progression from FIGS. 15-18, moving the container 100 toward the open configuration decreases the angle between the lid front panel 126 and the lid outer panel 124 at the front edge 130 of the lid outer panel 124 and increases the co-interior angle between the lid front panel 126 and the lid inner panel 122 at the front edge 128 of the lid inner panel 122. As shown, the same movement increases the angle between the lid back panel 132 and the lid outer panel 124 at the back edge 136 of the lid outer panel 124 and decreases the co-interior angle between the lid back panel 132 and the lid inner panel 122 at the back edge 134 of the lid inner panel 122.

With respect to the base portion 150, as shown in the progression from FIGS. 15-18, moving the container 100 toward the open configuration decreases the angle between the base front panel 156 and the base outer panel 154 at the front edge 160 of the base outer panel 154 and increases the co-interior angle between the base front panel 156 and the base inner panel 152 at the front edge 158 of the base inner panel 152. As shown, the same movement increases the angle between the base back panel 162 and the base outer panel 154 at the back edge 166 of the base outer panel 154 and decreases the co-interior angle between the base back panel 162 and the base inner panel 152 at the back edge 164 of the base inner panel 152.

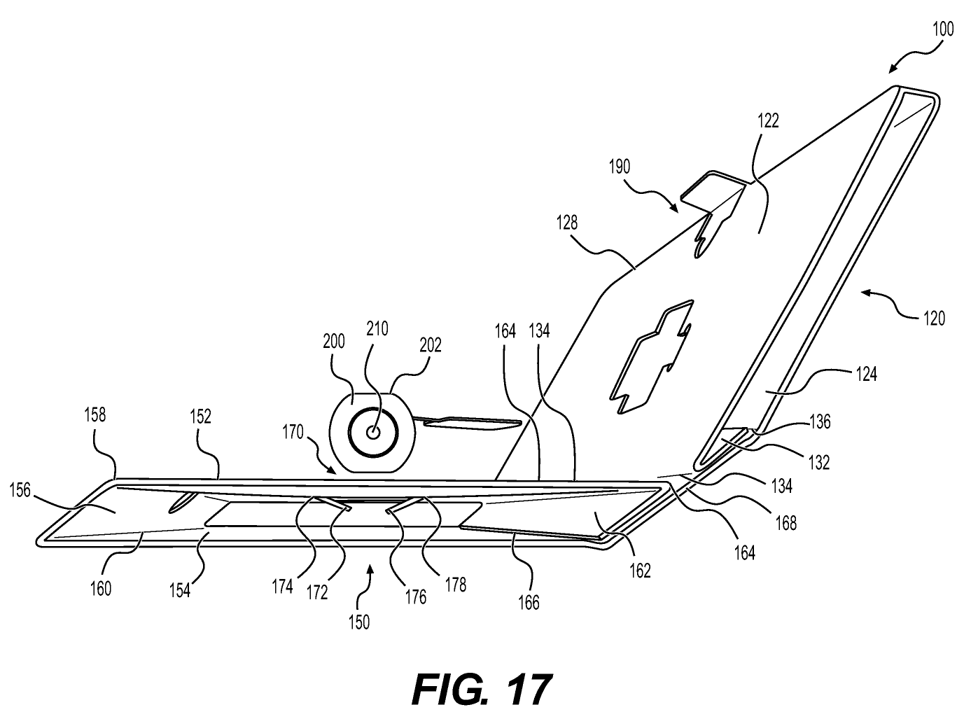

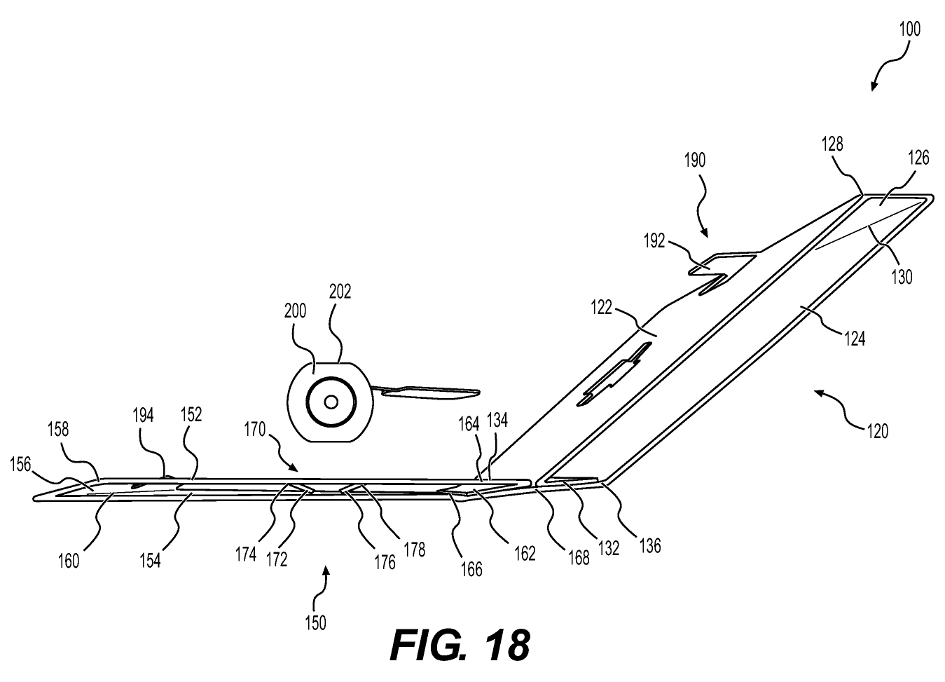

As progressively shown in FIGS. 15-18, and described herein, when the container 100 moves from the closed configuration toward the open configuration, various panels pivot and angles change such that spacings between the lid outer panel 124 and the lid inner panel 122 and between the base outer panel 154 and the base inner panel 152 decrease. Accordingly, as shown in FIG. 17 and further in FIG. 18, moving the container 100 from the closed configuration toward the open configuration automatically increases the spacing between a top surface of the medical device 200 and the base inner panel 152, thereby facilitating dispensing of the medical device 200 from the opening 170/container 100.

### Method of Dispensing a Medical Device

In general, the present disclosure is directed to a container 100 for a medical device 200 as well as methods of preparing and using the same. Specifically, the present disclosure is further directed to a method of dispensing a medical device 200 from a container 100. As described herein, the container 100 of any of the previous examples is movable from a closed configuration toward and open configuration, and vice-a-versa. As further described herein, the container 100 may be configured to dispense a medical device 200 either automatically upon movement of the container 100 toward the open configuration or may be configured to dispense the medical device 200 thereafter upon additional movement of the container 100.

In some examples, the method first includes providing a container with a medical device 200 retained therein, where the container may be container 100 of any of the previous examples, such as in FIGS. 1-19. The method then includes moving the container 100 from the closed configuration where the lid inner panel 122 is positioned adjacent to the base inner panel 152 toward the open configuration where the lid inner panel 122 is spaced apart from the base inner panel 152. Finally, the medical device 200 is dispensed from the container 100 by decreasing a spacing between the base inner panel 152 and the base outer panel 154 such that the medical device 200 is removed from the base opening 170. In some examples the first tab and second tabs 172, 176 urge the medical device 200 upward and away from the base outer panel 154. Decreasing the spacing between the base outer panel 154 and the base inner panel 152, as described herein, causes the base outer panel 154 to exert an upward force on a portion of the medical device 200 and/or the first tab 172 or the second tab 176 such that the spacing between a top surface 202 of the medical device 200 and the base inner panel 152 is increased, thereby releasing the medical device 200 from the base opening 170.

In some examples, the base inner panel 152 further includes a first locking tab 171 extending from a first locking side edge 173 of the base opening 170 and a second locking tab 177 extending from a second locking side edge 179 of the base opening 170. In such examples, retaining the medical device 200 within the base opening 170 further includes engaging the first locking tab 171 and/or the second locking tab 177 with the medical device 200. The first and second locking tabs 171, 177 provide an inwardly directed (e.g., directed toward a center of the base opening) and downwardly directed (e.g., directed toward the base outer panel 154) forces holding the medical device 200 within the base opening 170. In such examples, when the base outer panel 154 exerts an upward force on a portion of the medical device 200, the first tab 172 and/or the second tab 176, the medical device 200 in turn exerts an upward force against the first and second locking tabs 171, 177 until the locking tabs 171, 177 bend or are otherwise displaced and the medical device 200 is released from within the base opening 170.

In some examples, the method of moving the container 100 from the closed configuration to the open configuration further includes disengaging the engagement mechanism 190, thereby increasing a spacing between the lid portion 120 and the base portion 150. As shown, for example, in the progression between FIGS. 3-4, the engagement mechanism engages the lid portion 120 with the base portion 150 in the closed configuration and disengages the lid portion 120 from the base portion 150 in the open configuration, thereby permitting the spacing between the lid portion 120 and base portion 150 to increase.

In some examples, the medical device 200 includes a central opening 210 extending therethrough. As illustrated in FIG. 8, in the open configuration, the central opening 210 is provided above the base inner panel 152. This allows an ancillary medical device, such as a catheter or needle, to be coupled to the central opening 210 while the medical device 200 is secured within the container 100/base opening 170.

### Method of Dispensing Specific to Primary Embodiment

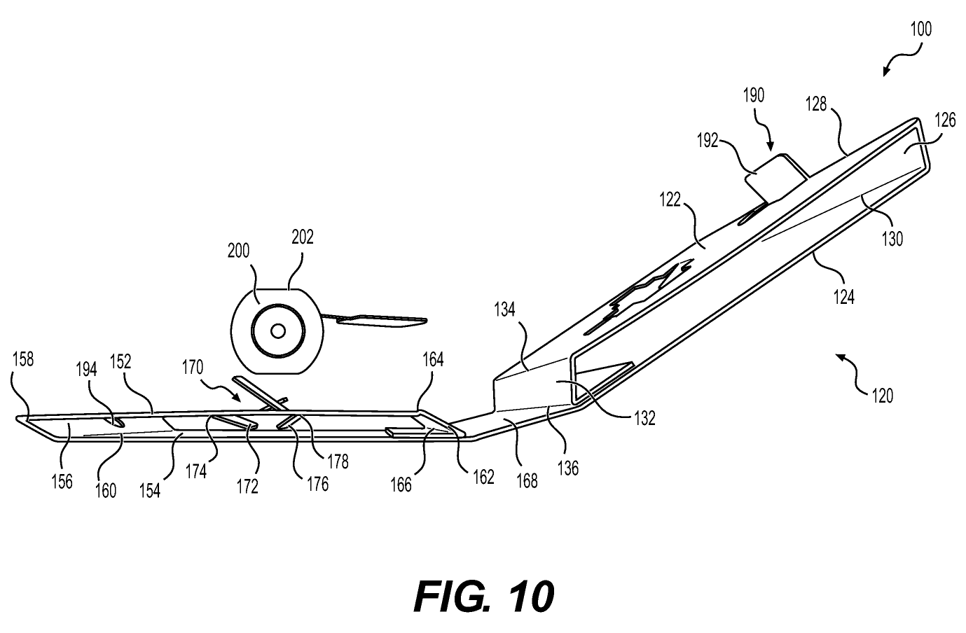

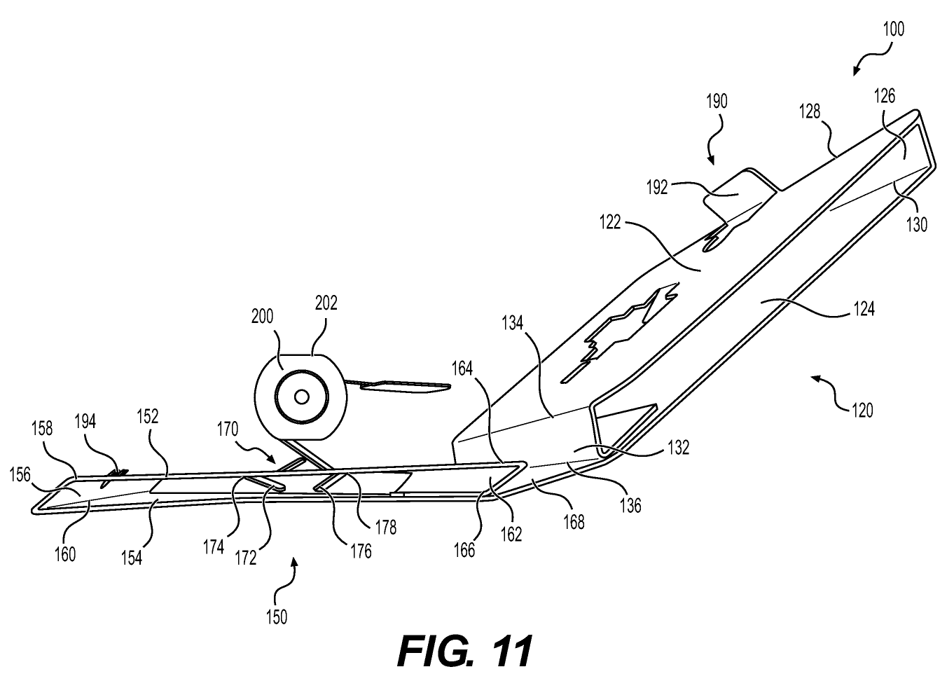

In the example container 100 shown in FIGS. 8-11, dispensing the medical device 200 from the container 100 further includes pivoting the base front panel 156 with respect to the base outer panel 154 at the front edge 160 of the base outer panel 154 and pivoting the base back panel 162 with respect to the base outer panel 154 at the back edge 166 of the base outer panel 154. As shown in FIGS. 10 and 11, the base front panel 156 and base back panel 162 may pivot along front edges 158 and 160 and back edges 164 and 166, respectively, in various angles to decrease the spacing between the base outer panel 154 and the base inner panel 152 so as to increase the spacing between the top surface 202 of the medical device 200 and the base inner panel 152.

Specifically, as shown in the progression from FIGS. 8-10, dispensing the medical device 200 from the container 100 may include increasing the angle between the base front panel 156 and the base outer panel 154 at the front edge 160 of the base outer panel 154 and decreasing the co-interior angle between the base front panel 156 and the base inner panel 152 at the front edge 158 of the base inner panel 152. As shown, the same movement decreases the angle between the base back panel 162 and the base outer panel 154 at the back edge 166 of the base outer panel 154 and increases the co-interior angle between the base back panel 162 and the base inner panel 152 at the back edge 164 of the base inner panel 152.

Alternatively, as shown in the progression from FIG. 8 to FIG. 11, dispensing the medical device 200 from the container 100 may include decreasing the angle between the base front panel 156 and the base outer panel 154 at the front edge 160 of the base outer panel 154 and increasing the co-interior angle between the base front panel 156 and the base inner panel 152 at the front edge 158 of the base inner panel 152. As shown, the same movement increases the angle between the base back panel 162 and the base outer panel 154 at the back edge 166 of the base outer panel 154 and decreases the co-interior angle between the base back panel 162 and the base inner panel 152 at the back edge 164 of the base inner panel 152.

### Method of Dispensing Specific to Secondary Embodiment

In another example container 100 as shown in FIGS. 15-18, moving the container 100 from the closed configuration to the open configuration, automatically decreases a spacing between the base outer panel 154 and the base inner panel 152, thereby releasing the medical device 200 from the base opening 170.

### Method for packaging and sterilizing a medical device

The present disclosure is further directed to a method for packaging and sterilizing a medical device 200 and container 100.

In some examples, the method includes the steps of: (1) providing a container 100 comprising: a lid portion 120 comprising: a lid inner panel 122; a lid outer panel 124, spaced apart from and opposing the lid inner panel 122; a lid front panel 126 extending between a front edge 128 of the lid inner panel 122 and a front edge 130 of the lid outer panel 124; a lid back panel 132 extending from a back edge 134 of the lid inner panel 122 toward a back edge 136 of the lid outer panel 124; and a base portion 150 pivotably coupled to the lid portion 120, the base portion 150 comprising: a base inner panel 152; a base outer panel 154, spaced apart from and opposing the base inner panel 152; a base front panel 156 extending between a front edge 158 of the base inner panel 152 and a front edge 160 of the base outer panel 154; and a base back panel 162 extending from a back edge 164 of the base inner panel 152 toward a back edge 166 of the base outer panel; and a joining panel 168 extending between the back edge 136 of the lid outer panel 124 and the back edge 166 base outer panel 154, wherein the lid portion 120 and/or the base portion 150 are moveable between a closed configuration and an open configuration, where in the closed configuration the lid inner panel 122 is positioned adjacent to the base inner panel 152, and in the open configuration the lid inner panel 122 is spaced apart from the base inner panel 152; (2) with the lid portion 120 and/or the base portion 150 in an open configuration, inserting a medical device 200 into a base opening 170 provided in the base inner panel 152; (3) retaining the medical device 200 with the base opening 170 by engaging a first tab 172 extending from a first side edge 174 of the base opening 170 with the medical device 200, the first tab 172 providing an inwardly directed (e.g., directed toward the center of the base opening) force and an upwardly directed (e.g., directed toward the base inner panel 152) force against the medical device 200; (4) moving the container 100 into a closed configuration; (5) inserting into the container 100 into a pouch; (6) sealing the pouch; and (7) sterilizing the pouch.

In some examples, the method further includes inserting the pouch into an outer container. In some examples, the outer container 100 can include a plurality of pouches, for example, 10 pouches per container.

These and other features, aspects, and advantages of the present disclosure will become better understood with reference to the following description and appended claims. The accompanying drawings, which are incorporated in and constitute part of this specification, illustrate examples of the claimed invention and, together with the description, serve to explain the principles of the disclosure.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

As used in the specification and the appended claims, the singular forms “a,” “an” and “the” include plural referents unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: A system for containing a medical device, the system comprising a container comprising: a lid portion comprising: a lid inner panel; a lid outer panel spaced apart from and opposing the lid inner panel; a lid front panel extending between a front edge of the lid inner panel and a front edge of the lid outer panel; a lid back panel extending from a back edge of the lid inner panel toward a back edge of the lid outer panel; a base portion pivotably coupled to the lid portion, the base portion comprising: a base inner panel; a base outer panel spaced apart from and opposing the base inner panel; a base front panel extending between a front edge of the base inner panel and a front edge of the base outer panel; a base back panel extending from a back edge of the base inner panel toward a back edge of the base outer panel; and a joining panel extending between the back edge of the lid outer panel and the back edge of the base outer panel; wherein the container is movable between a closed configuration and an open configuration, where in the closed configuration the lid inner panel is positioned adjacent to the base inner panel, and in the open configuration the lid inner panel is spaced apart from the base inner panel.

Example 2: A system according to any example herein, particularly example 1, wherein, in the closed configuration, the lid back panel is coplanar with the base back panel.

Example 3: A system according to any example herein, particularly examples 1-2, wherein, in the closed configuration, each of the lid back panel and base back panel are substantially parallel with the joining panel.

Example 4: A system according to any example herein, particularly examples 1-3, wherein, in the closed configuration, each of the lid back panel and base back panel are in contact with the joining panel.

Example 5: A system according to any example herein, particularly examples 1-4, wherein, in the closed configuration, the back edge of the lid inner panel is colinear with the back edge of the base inner panel.

Example 6: A system according to any example herein, particularly examples 1-5, wherein, in the closed configuration, the back edge of the lid inner panel and the back edge of the base inner panel are in contact with the joining panel.

Example 7: A system according to any example herein, particularly examples 1-6, wherein, in the closed configuration, the lid front panel is coplanar with the base front panel.

Example 8: A system according to any example herein, particularly examples 1-7, wherein, in the closed configuration, the lid outer panel is substantially parallel with the base outer panel.

Example 9: A system according to any example herein, particularly examples 1-8, wherein, in the open configuration, the lid front panel is not coplanar with the base front panel.

Example 10: A system according to any example herein, particularly examples 1-9, wherein, in the open configuration, the lid outer panel is not parallel with the base outer panel.

Example 11: A system according to any example herein, particularly examples 1-10, wherein, when the container moves from the closed configuration toward the open configuration: an angle between the lid outer panel and the joining panel increases, and an angle between the base outer panel and the joining panel increases.

Example 12: A system according to any example herein, particularly examples 1-11, wherein, when the container moves between the closed configuration and the open configuration: the lid back panel pivots with respect to the joining panel at the back edge of the lid outer panel; and the base back panel pivots with respect to the joining panel at the back edge of the base outer panel.

Example 13: A system according to any example herein, particularly examples 1-12, wherein, when the container moves from the closed configuration toward the open configuration: an angle between the lid back panel and the joining panel increases, and an angle between the base back panel and the joining panel increases.

Example 14: A system according to any example herein, particularly examples 1-13, wherein, in the open configuration, the lid back panel and base back panel are not coplanar.

Example 15: A system according to any example herein, particularly examples 1-14, wherein, in the open configuration, neither the lid back panel nor the base back panel are substantially parallel with the joining panel.

Example 16: A system according to any example herein, particularly examples 1-15, wherein, in the open configuration, neither the back edge of the lid inner panel and the back edge of the base inner panel are in contact with the joining panel.

Example 17: A system according to any example herein, particularly examples 1-16, wherein, in the open configuration, the back edge of the lid inner panel and the back edge of the base inner panel are spaced apart.

Example 18: A system according to any example herein, particularly examples 1-17, wherein, when in the open configuration: the lid inner panel and the lid outer panel are spaced apart; and the base inner panel and the base outer panel are spaced apart.

Example 19: A system according to any example herein, particularly examples 1-18, wherein, in both the open and closed configuration, the lid back panel is coplanar with the base back panel.

Example 20: A system according to any example herein, particularly examples 1-11 and 19, wherein, in both the open and closed configuration, each of the lid back panel and base back panel are substantially parallel with the joining panel.

Example 21: A system according to any example herein, particularly examples 1-11 and 19-20, wherein, in both the open and closed configuration, each of the lid back panel and base back panel are in contact with the joining panel.

Example 22: A system according to any example herein, particularly examples 1-11 and 19-21, wherein, in both the open and closed configuration, the back edge of the lid inner panel is colinear with the back edge of the base inner panel.

Example 23: A system according to any example herein, particularly examples 1-11 and 19-22, wherein, in both the open and closed configuration, each of the back edge of the lid inner panel and the back edge of the base inner panel are in contact with the joining panel.

Example 24: A system according to any example herein, particularly examples 1-11 and 19-23, wherein, when the container moves from the closed configuration toward the open configuration, a spacing between the lid outer panel and the lid inner panel decreases.

Example 25: A system according to any example herein, particularly examples 1-11 and 19-24, wherein, when the container moves from the closed configuration toward the open configuration, a spacing between the base outer panel and the base inner panel decreases.

Example 26: A system according to any example herein, particularly examples 1-11 and 19-25, wherein, when the container moves between the closed configuration and the open configuration, the lid inner panel and the base inner panel pivot with respect to each other along the back edge of the lid inner panel and the back edge of the base inner panel, wherein the back edge of the lid inner panel and the back edge of the base inner panel are colinear.

Example 27: A system according to any example herein, particularly examples 1-11 and 19-26, wherein, when the container moves between the closed configuration and the open configuration, the lid front panel pivots with respect to the lid inner panel and the lid outer panel.

Example 28: A system according to any example herein, particularly examples 1-11 and 19-27, wherein, when the container moves between the closed configuration and the open configuration, the base front panel pivots with respect to the base inner panel and the base outer panel.

Example 29: A system according to any example herein, particularly examples 1-11 and 19-28, wherein, when in the open configuration such that the lid outer panel and base outer panel are coplanar: the lid inner panel is positioned adjacent the lid outer panel; and the base inner panel is positioned adjacent the base outer panel.

Example 30: A system according to any example herein, particularly examples 1-29, wherein, when the container moves from the closed configuration toward the open configuration: an angle between the lid front panel and the lid outer panel at the front edge of the lid outer panel decreases, and an angle between the lid front panel and the lid inner panel at the front edge of the lid inner panel increases.

Example 31: A system according to any example herein, particularly examples 1-30, wherein, when the container moves from the closed configuration toward the open configuration: an angle between the lid back panel and the lid outer panel at the back edge of the lid outer panel increases, and an angle between the lid back panel and the lid inner panel at the back edge of the lid inner panel decreases.

Example 32: A system according to any example herein, particularly examples 1-31, wherein, when the container moves from the closed configuration toward the open configuration: an angle between the base front panel and the base outer panel at the front edge of the base outer panel decreases, and an angle between the base front panel and the base inner panel at the front edge of the base inner panel increases.

Example 33: A system according to any example herein, particularly examples 1-32, wherein, when the container moves from the closed configuration toward the open configuration: an angle between the base back panel and the base outer panel at the back edge of the base outer panel increases, and an angle between the base back panel and the base inner panel at the back edge of the base inner panel decreases.

Example 34: A system according to any example herein, particularly examples 1-33, wherein the base inner panel defines a base opening sized and configured to receive a medical device, the base inner panel comprising a first tab extending from a first side edge of the base opening and a second tab extending from a second side edge of the base opening, wherein the first tab and the second tab extend from the base inner panel in a direction toward the base outer panel.

Example 35: A system according to any example herein, particularly example 33, further comprising a medical device, wherein the first tab and the second tab of the base inner panel provide an inwardly directed force and an upwardly directed force, thereby holding the medical device within the base opening and urging the medical device away from the base outer panel.

Example 36: A system according to any example herein, particularly examples 1-35, wherein the base inner panel comprises a first locking tab extending from a first locking side edge of the base opening, wherein the first locking tab extends from the base inner panel in a direction toward the lid inner panel.

Example 37: A system according to any example herein, particularly example 36, wherein the base inner panel comprises a second locking tab extending from a second locking side edge of the base opening, wherein the second locking tab extends from the base inner panel in a direction toward the lid inner panel.

Example 38: A system according to any example herein, particularly example 36, wherein the first locking side edge is on an opposite side of the base opening relative to the second locking side edge.

Example 39: A system according to any example herein, particularly example 36, further comprising a medical device, wherein the first locking tab and the second locking tab provide an inwardly directed force and a downwardly directed force, thereby holding the medical device within the base opening.

Example 40: A system according to any example herein, particularly examples 1-39, wherein the medical device comprises at least one of a medical device accessory.

Example 41: A system according to any example herein, particularly examples 1-40, wherein the base inner panel defines a first finger opening adjacent the first side edge or second side edge.

Example 42: A system according to any example herein, particularly example 41, wherein a portion of the first finger opening extends beyond the first side edge or second side edge and into the first tab or second tab.

Example 43: A system according to any example herein, particularly examples 1-42, wherein the lid inner panel defines a lid opening sized and configured to receive a medical device, the lid inner panel comprising a first tab extending from a first side edge of the lid opening and a second tab extending from a second side edge of the lid opening, wherein the first tab and the second tab extend from the lid inner panel in a direction toward the lid outer panel.

Example 55: A system according to any example herein, particularly example 43, further comprising a medical device, wherein the first tab and the second tab of the lid inner panel provide an inwardly directed force and a downwardly directed force, thereby holding the medical device within the lid opening and urging the medical device away from the lid outer panel.

Example 45: A system according to any example herein, particularly examples 43-44, wherein the lid inner panel defines a second finger opening adjacent the first side edge or second side edge.

Example 46: A system according to any example herein, particularly examples 43-45, wherein a portion of the second finger opening extends beyond the first side edge or second side edge and into the first tab or second tab.

Example 47: A method of dispensing a medical device from a container comprising: (1) providing a container with a medical device retained therein, the container comprising: a lid portion comprising: a lid inner panel; a lid outer panel spaced apart from and opposing the lid inner panel; a lid front panel extending between a front edge of the lid inner panel and a front edge of the lid outer panel; and a lid back panel extending from a back edge of the lid inner panel toward a back edge of the lid outer panel; a base portion pivotably coupled to the lid portion, the base portion comprising: a base inner panel defining a base opening sized and configured to receive the medical device; a base outer panel spaced apart from and opposing the base inner panel; a base front panel extending between a front edge of the base inner panel and a front edge of the base outer panel; and a base back panel extending from a back edge of the base inner panel toward a back edge of the base outer panel; and a joining panel extending between the back edge of the lid outer panel and the back edge base outer panel; (2) moving container from a closed configuration toward an open configuration, where in the closed configuration the lid inner panel is positioned adjacent to the base inner panel, and in the open configuration the lid inner panel is spaced apart from the base inner panel; and (3) decreasing a spacing between the base outer panel and the base inner panel such that a spacing between the top surface of the medical device and the base inner panel increases.

Example 78: A method according to any example herein, particularly example 47, wherein the base inner panel further comprises a first tab extending from a first side edge of the base opening and a second tab extending from a second side edge of the base opening, wherein, when the container moves from the closed configuration toward the open configuration, the first and second tabs urge the medical device away from the base outer panel, wherein, decreasing a spacing between the base outer panel and the base inner panel causes the base outer panel to exert an upward force on a portion of the medical device and/or the first tab or the second tab such that the medical device is released from within the base opening.

Example 49: A method according to any example herein, particularly example 47, wherein the base inner panel further comprises a first locking tab extending from a first locking side edge of the base opening and a second locking tab extending from a second locking side edge of the base opening, wherein, when the base outer panel exerts an upward force on a portion of the medical device and/or the first tab or the second tab, the medical device exerts an upward force against the first locking tab and the second locking tab, and the medical device is released from within the base opening.

Example 50: A method according to any example herein, particularly examples 47-49, wherein dispensing the medical device from the container further comprises: pivoting the lid back panel with respect to the joining panel at the back edge of the lid outer panel; and pivoting the base back panel with respect to the joining panel at the back edge of the base outer panel.

Example 51: A method according to any example herein, particularly example 50, wherein, in the open configuration, a spacing between the base outer panel and the base inner panel decreases and the medical device is released from the base opening.

Example 52: A method according to any example herein, particularly examples 47-51, wherein the medical device comprises a central opening extending therethrough, wherein, in the open configuration, the central opening is provided above the base inner panel.

Example 53: A method for packaging and sterilizing a medical device and container, the method comprising the steps of: (1) providing a container comprising: a lid portion comprising: a lid inner panel; a lid outer panel spaced apart from and opposing the lid inner panel; a lid front panel extending between a front edge of the lid inner panel and a front edge of the lid outer panel; a lid back panel extending from a back edge of the lid inner panel toward a back edge of the lid outer panel; and a base portion pivotably coupled to the lid portion, the base portion comprising: a base inner panel; a base outer panel spaced apart from and opposing the base inner panel; a base front panel extending between a front edge of the base inner panel and a front edge of the base outer panel; and a base back panel extending from a back edge of the base inner panel toward a back edge of the base outer panel; and a joining panel extending between the back edge of the lid outer panel and the back edge base outer panel, wherein the lid portion and/or the base portion are moveable between a closed configuration and an open configuration, where in the closed configuration the lid inner panel is positioned adjacent to the base inner panel, and in the open configuration the lid inner panel is spaced apart from the base inner panel; (2) with the lid portion and/or the base portion in an open configuration, inserting a medical device into a base opening provided in the base inner panel; (3) retaining the medical device with the base opening by engaging a first tab extending from a first side edge of the base opening with the medical device, the first tab providing an inwardly directed force and an upwardly directed force against the medical device; (4) moving the container into a closed configuration; (5) inserting into the container into a pouch; (6) sealing the pouch; and (7) sterilizing the pouch.

Example 54: A method according to any example herein, particularly example 53, wherein retaining the medical device with the base opening further comprises engaging a first locking tab extending from a first locking side edge of the base opening with the medical device, the first locking tab providing an inwardly directed force and a downwardly directed force, thereby holding the medical device within the base opening.

Example 55: A method according to any example herein, particularly example 54, wherein retaining the medical device with the base opening further comprises engaging a second locking tab extending from a second locking side edge of the base opening with the medical device, the second locking tab providing an inwardly directed (e.g., directed toward a center of the base opening) force and a downwardly directed (e.g., directed toward the base outer panel) force, thereby holding the medical device within the base opening.

Example 56: A method according to any example herein, particularly examples 53-55, further comprising: inserting the pouch into an outer container.

Example 57: A method according to any example herein, particularly example 56, wherein moving the container into the closed configuration comprises securing the container in the closed configuring using an engagement mechanism between the lid portion and the base portion.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. A system for containing a medical device, the system comprising a container comprising:
a lid portion comprising:
a lid inner panel;
a lid outer panel spaced apart from and opposing the lid inner panel;
a lid front panel extending between a front edge of the lid inner panel and a front edge of the lid outer panel;
a lid back panel extending from a back edge of the lid inner panel toward a back edge of the lid outer panel;
a base portion pivotably coupled to the lid portion, the base portion comprising:
a base inner panel;
a base outer panel spaced apart from and opposing the base inner panel;
a base front panel extending between a front edge of the base inner panel and a front edge of the base outer panel;
a base back panel extending from a back edge of the base inner panel toward a back edge of the base outer panel; and
a joining panel extending between the back edge of the lid outer panel and the back edge of the base outer panel;
wherein the container is movable between a closed configuration and an open configuration, where in the closed configuration the lid inner panel is positioned adjacent to the base inner panel, and in the open configuration the lid inner panel is spaced apart from the base inner panel.

2. The system of claim 1, wherein, in the closed configuration:
the lid back panel is coplanar with the base back panel; and
each of the lid back panel and base back panel are substantially parallel with the joining panel.

3. The system of claim 1, wherein, in the closed configuration:
the back edge of the lid inner panel is colinear with the back edge of the base inner panel; and
the back edge of the lid inner panel and the back edge of the base inner panel are in contact with the joining panel.

4. The system of claim 1, wherein, in the closed configuration:
the lid front panel is coplanar with the base front panel;
the lid outer panel is substantially parallel with the base outer panel; and
the lid front panel is not coplanar with the base front panel.

5. The system of claim 1, wherein, when the container moves from the closed configuration toward the open configuration:
an angle between the lid outer panel and the joining panel increases;
an angle between the base outer panel and the joining panel increases;
an angle between the lid back panel and the joining panel increases; and
an angle between the base back panel and the joining panel increases.

6. The system of claim 1, wherein, when the container moves between the closed configuration and the open configuration:
the lid back panel pivots with respect to the joining panel at the back edge of the lid outer panel; and
the base back panel pivots with respect to the joining panel at the back edge of the base outer panel.

7. The system of claim 1, wherein, in the open configuration:
the lid back panel and base back panel are not coplanar;
neither the lid back panel nor the base back panel are substantially parallel with the joining panel.

8. The system of claim 1, wherein, in the open configuration:
the back edge of the lid inner panel and the back edge of the base inner panel are spaced apart;
the lid inner panel and the lid outer panel are spaced apart; and
the base inner panel and the base outer panel are spaced apart.

9. The system of claim 1, wherein, in both the open and closed configuration:
the lid back panel is coplanar with the base back panel;
both the open and closed configuration, each of the lid back panel and base back panel are substantially parallel with the joining panel;
each of the lid back panel and base back panel are in contact with the joining panel;
the back edge of the lid inner panel is colinear with the back edge of the base inner panel; and
each of the back edge of the lid inner panel and the back edge of the base inner panel are in contact with the joining panel.

10. The system of claim 1, wherein, when the container moves from the closed configuration toward the open configuration:
a spacing between the lid outer panel and the lid inner panel decreases;
an angle between the lid front panel and the lid outer panel at the front edge of the lid outer panel decreases;
an angle between the lid front panel and the lid inner panel at the front edge of the lid inner panel increases;
an angle between the lid back panel and the lid outer panel at the back edge of the lid outer panel increases, and
an angle between the lid back panel and the lid inner panel at the back edge of the lid inner panel decreases.

11. The system of claim 1, wherein, when the container moves from the closed configuration toward the open configuration:
a spacing between the base outer panel and the base inner panel decreases;
an angle between the base back panel and the base outer panel at the back edge of the base outer panel increases, and
an angle between the base back panel and the base inner panel at the back edge of the base inner panel decreases.

12. The system of claim 1, wherein, when the container moves between the closed configuration and the open configuration:
the lid inner panel and the base inner panel pivot with respect to each other along the back edge of the lid inner panel and the back edge of the base inner panel, wherein the back edge of the lid inner panel and the back edge of the base inner panel are colinear;
the lid front panel pivots with respect to the lid inner panel and the lid outer panel; and
the base front panel pivots with respect to the base inner panel and the base outer panel.

13. The system of claim 1, wherein the base inner panel defines a base opening sized and configured to receive a medical device, the base inner panel comprising a first tab extending from a first side edge of the base opening and a second tab extending from a second side edge of the base opening,
wherein the first tab and the second tab extend from the base inner panel in a direction toward the base outer panel.

14. The system of claim 13, further comprising a medical device, wherein the first tab and the second tab of the base inner panel provide an inwardly directed force and an upwardly directed force, thereby holding the medical device within the base opening and urging the medical device away from the base outer panel.

15. The system of claim 1, wherein the base inner panel comprises a first locking tab extending from a first locking side edge of the base opening and a second locking tab extending from a second locking side edge of the base opening,
wherein the first locking tab and the second locking tab each extend from the base inner panel in a direction toward the lid inner panel, and
wherein the first locking side edge is on an opposite side of the base opening relative to the second locking side edge.

16. The system of claims claim 15, further comprising a medical device,
wherein the first locking tab and the second locking tab provide an inwardly directed force and a downwardly directed force, thereby holding the medical device within the base opening.

17. The system of claim 1, wherein the lid inner panel defines a lid opening sized and configured to receive a medical device, the lid inner panel comprising a first tab extending from a first side edge of the lid opening and a second tab extending from a second side edge of the lid opening,
wherein the first tab and the second tab extend from the lid inner panel in a direction toward the lid outer panel.

18. The system of claim 17, further comprising a medical device, wherein the first tab and the second tab of the lid inner panel provide an inwardly directed force and a downwardly directed force, thereby holding the medical device within the lid opening and urging the medical device away from the lid outer panel.

19. A method of dispensing a medical device from a container comprising:
providing a container with a medical device retained therein, the container comprising:
a lid portion comprising:
a lid inner panel;
a lid outer panel spaced apart from and opposing the lid inner panel;
a lid front panel extending between a front edge of the lid inner panel and a front edge of the lid outer panel; and
a lid back panel extending from a back edge of the lid inner panel toward a back edge of the lid outer panel;
a base portion pivotably coupled to the lid portion, the base portion comprising:
a base inner panel defining a base opening sized and configured to receive the medical device;
a base outer panel spaced apart from and opposing the base inner panel;
a base front panel extending between a front edge of the base inner panel and a front edge of the base outer panel; and
a base back panel extending from a back edge of the base inner panel toward a back edge of the base outer panel; and
a joining panel extending between the back edge of the lid outer panel and the back edge base outer panel;
moving container from a closed configuration toward an open configuration, where in the closed configuration the lid inner panel is positioned adjacent to the base inner panel, and in the open configuration the lid inner panel is spaced apart from the base inner panel; and
decreasing a spacing between the base outer panel and the base inner panel such that a spacing between a top surface of the medical device and the base inner panel increases.

20. A method for packaging and sterilizing a medical device and container, the method comprising the steps of:
providing a container comprising:
a lid portion comprising:
a lid inner panel;
a lid outer panel spaced apart from and opposing the lid inner panel;
a lid front panel extending between a front edge of the lid inner panel and a front edge of the lid outer panel;
a lid back panel extending from a back edge of the lid inner panel toward a back edge of the lid outer panel; and
a base portion pivotably coupled to the lid portion, the base portion comprising:
a base inner panel;
a base outer panel spaced apart from and opposing the base inner panel;
a base front panel extending between a front edge of the base inner panel and a front edge of the base outer panel; and
a base back panel extending from a back edge of the base inner panel toward a back edge of the base outer panel; and
a joining panel extending between the back edge of the lid outer panel and the back edge base outer panel,
wherein the lid portion and/or the base portion are moveable between a closed configuration and an open configuration, where in the closed configuration the lid inner panel is positioned adjacent to the base inner panel, and in the open configuration the lid inner panel is spaced apart from the base inner panel;
with the lid portion and/or the base portion in an open configuration, inserting a medical device into a base opening provided in the base inner panel;
retaining the medical device with the base opening by engaging a first tab extending from a first side edge of the base opening with the medical device, the first tab providing an inwardly directed force and an upwardly directed force against the medical device;
moving the container into a closed configuration;
inserting into the container into a pouch;
sealing the pouch; and
sterilizing the pouch.
